(19) United States
(12) Patent Application Publication
(10) Pub. No.: US 2019/0057768 A1
(43) Pub. Date: Feb. 21, 2019

(54) INFORMATION PROCESSING APPARATUS, METHOD THEREFOR, AND STORAGE MEDIUM

(71) Applicant: CANON KABUSHIKI KAISHA, Tokyo (JP)

(72) Inventor: Masahiro Abe, Yamato-shi (JP)

(21) Appl. No.: 16/079,636

(22) PCT Filed: Feb. 28, 2017
(86) PCT No.: PCT/JP2017/007944

§ 371 Date: Aug. 24, 2018

(30) Foreign Application Priority Data
Mar. 3, 2016 (JP) 2016-041488

(51) Int. Cl. G16H 30/20; G16H 40/63; G06Q 10/00; G16H 10/60

(52) U.S. Cl. CPC G16H 30/20; G16H 10/60; G06Q 10/00; G16H 40/63

(57) ABSTRACT

An information processing apparatus includes: first display control means for causing display means to display inspection information including a test image in a selectable form; second display control means for causing the display means to display inspection request information including specifying information for specifying an imaging technique in a selectable form; determination means for determining necessity of an additional inspection by comparing an imaging technique for a test image included in inspection information selected on display performed by the first display control means with an imaging technique specified by the specifying information included in inspection request information selected on display performed by the second display control means; and third display control means for causing the display means to display information indicating that an additional inspection is required, when the determination means determines that an additional inspection is required.

### TECHNICAL FIELD

The present invention relates to an information processing apparatus, a method therefor, and a storage medium and, more particularly, to control of medical inspections.

### BACKGROUND ART

Recently, hospital information systems using network connections have been constructed in hospitals. When, for example, performing X-ray imaging, a doctor inputs an inspection instruction with a terminal and transmits the instruction to an RIS (Radiology Information System) in a radiology department as a request destination.

This request information includes a department name as a request source, inspection items, and individual data of a patient as an imaging target. A radiation imaging system receives the inspection request information from the RIS and executes X-ray imaging in accordance with the request content. In general, each obtained image is provided with inspection information and transferred to the storage in a PACS (Picture Archiving and Communication System) or printed out.

The radiological technician using the radiation imaging system selects the inspection request information of a target object from the inspection request information list received from external equipment such as an RIS, and repeats imaging based on the selected inspection request information. There is known an arrangement configured to change an inspection order when the technician has started an inspection upon selecting wrong inspection request information and allow him/her to easily change the inspection order in consideration of the inheritance of obtained images, consistency with inspection execution information, and the like (see PTL 1).

### CITATION LIST

### Patent Literature

[PTL 1] Japanese Patent Laid-Open No. 2013-215274

### SUMMARY OF INVENTION

### Technical Problem

When the operator notices an error after transmitting an image to a PACS or the like upon completion of an inspection, he/she needs to output again an image with inspection information which should have been obtained or perform an inspection upon notifying that the inspection information which has already been output is invalid. At this time, if the test image obtained by the wrong execution can be used as a test image which should have been obtained, it is preferable to use the test image without executing any re-inspection.

It is, however, difficult for the operator of the conventional arrangement to easily grasp whether a test image obtained by wrong execution can be used as a test image which should have been obtained or additional imaging needs to be performed. For this reason, the operator needs to grasp, through a manual operation, imaging which needs to be additionally performed by, for example, visually checking an image wrongly obtained and inspection contents which should have been executed. In addition, the operator must manually correct the inspection information of the wrongly obtained test image. This has imposed more than a little operation load on the operator.

The present invention has been made in consideration of the above problem and provides a technique capable of easily checking and correcting an error if it is included in an inspection performed in accordance with an inspection request.

### Solution to Problem

According to one aspect of the present invention, there is provided an information processing apparatus which controls a medical inspection based on inspection request information received from external equipment, the apparatus comprising: first display control means for causing display means to display inspection information including a test image in a selectable form, which is transmitted to the external equipment; second display control means for causing the display means to display inspection request information including specifying information for specifying an imaging technique in a selectable form, which is received from the external equipment; determination means for determining necessity of an additional inspection by comparing an imaging technique for a test image included in inspection information selected on display performed by the first display control means with an imaging technique specified by the specifying information included in inspection request information selected on display performed by the second display control means; and third display control means for causing the display means to display information indicating that an additional inspection is required, when the determination means determines that an additional inspection is required.

According to another aspect of the present invention, there is provided an information processing apparatus which controls a medical inspection based on inspection request information received from external equipment, the apparatus comprising: first display control means for causing display means to display inspection information including a test image in a selectable form, which is transmitted to the external equipment; second display control means for causing the display means to display inspection request information including specifying information for specifying an imaging technique in a selectable form, which is received from the external equipment; determination means for determining necessity of an additional inspection by comparing an imaging technique for a test image included in inspection information selected on display performed by the first display control means with an imaging technique specified by the specifying information included in inspection request information selected on display performed by the second display control means; and transmission means for transmitting, to the external equipment, invalidity information for invalidating inspection information which corresponds to the selected inspection information and is managed by the external equipment, if the determination means determines that an additional inspection is required.

According to still another aspect of the present invention, there is provided an information processing method for an information processing apparatus which controls a medical inspection based on inspection request information received from external equipment, the method comprising: causing first display control means for causing display means to display inspection information including a test image in a selectable form, which is transmitted to the external equipment; causing second display control means for causing the display means to display inspection request information including specifying information for specifying an imaging technique in a selectable form, which is received from the external equipment; causing determination means for determining necessity of an additional inspection by comparing an imaging technique for a test image included in inspection information selected on display performed in the causing the first display control means with an imaging technique specified by the specifying information included in inspection request information selected on display performed in the causing the second display control means; and causing third display control means for causing the display means to display information indicating that an additional inspection is required, when it is determined in the causing the determination means that an additional inspection is required.

### Advantageous Effects of Invention

The present invention can provide a technique capable of easily checking and correcting an error if it is included in an inspection performed in accordance with an inspection request.

Further features of the present invention will become apparent from the following description of exemplary embodiments (with reference to the attached drawings).

### BRIEF DESCRIPTION OF DRAWINGS

FIG. 1 is a block diagram showing the hardware arrangement of a radiation imaging system.

FIG. 2 is a block diagram showing the functional arrangement of the radiation imaging system.

FIG. 3 is a flowchart showing a processing procedure executed by the radiation imaging system.

FIG. 4 is a view showing an example of a GUI in the radiation imaging system.

FIG. 5 is a view showing an example of a GUI in the radiation imaging system.

FIG. 6 is a view showing an example of a GUI in the radiation imaging system.

FIG. 7 is a view showing an example of a GUI in the radiation imaging system.

FIG. 8 is a view showing an example of a GUI in the radiation imaging system.

FIG. 9 is a view showing an example of a GUI in the radiation imaging system.

### DESCRIPTION OF EMBODIMENTS

The embodiments of the present invention will be described in detail below with reference to the accompanying drawings.

### First Embodiment

(Hardware Arrangement)

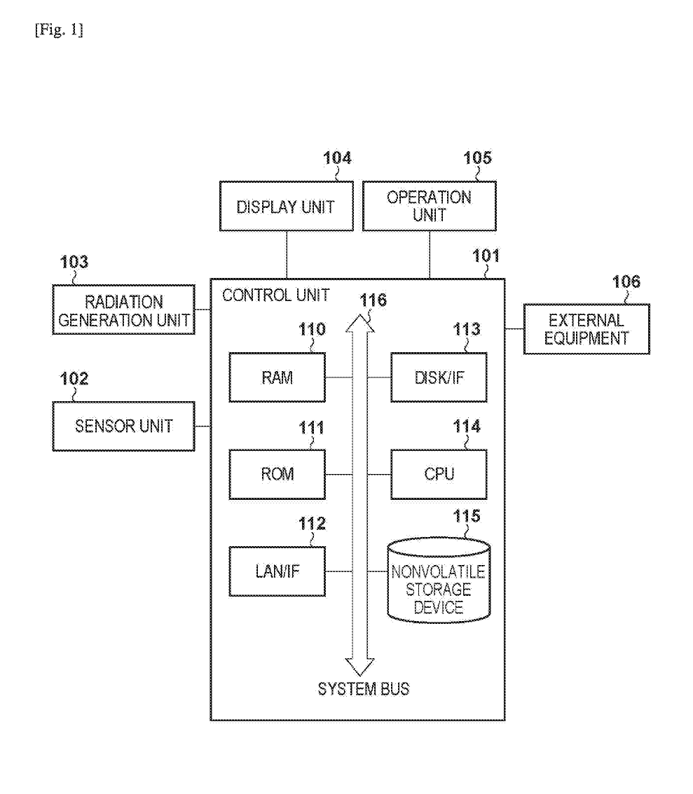

FIG. 1 shows an example of the hardware arrangement of a radiation imaging system according to an embodiment. As shown in FIG. 1, the radiation imaging system includes, for example, a control unit 101, a sensor unit 102, a radiation generation unit 103, a display unit 104, and an operation unit 105. Note that the respective functional units may be discrete devices respectively having different pieces of hardware or a plurality of functional units may be included in one device. In addition, radiation can be, for example, X-rays.

The control unit 101 is a control apparatus which drives the sensor unit and controls the X-ray generation unit based on inputs from the operator. The control unit 101 also manages various types of data such as sensor correction data, radiation imaging conditions, and image data. The control unit 101 includes constituent elements including, for example, a RAM (Random Access Memory) 110, a ROM (Read Only Memory) 111, a LAN/IF 112, a DISK/IF 113, a CPU 114, and a nonvolatile storage device 115 such as a hard disk. These pieces of hardware are connected to each other via a system bus 116. That is, the control unit 101 has the same arrangement as that of a general computer (information processing apparatus) such as a PC (Personal Computer).

The sensor unit 102 is a sensor unit which images a radiation signal emitted from the radiation generation unit 103 and transmitted through an object, and transfers the acquired image to the control unit 101. The radiation generation unit 103 is a radiation generation apparatus, which irradiates an object (not shown) and the sensor unit 102 with radiation based on an instruction from the control unit 101.

The display unit 104 is formed from a general monitor such as a CRT or liquid crystal display, and displays image data, a GUI (graphical User Interface), and the like on a screen. The operation unit 105 is formed from input devices such as a pointing device, a keyboard, and an irradiation switch. The operator (user) uses the operation unit 105 to input various types of commands and data to the control unit 101. Note that it is possible to use a monitor having the functions of both the display unit 104 and the operation unit 105, such as a touch panel.

External equipment 106 is an external system such as an image saving system which saves an image transferred from the control unit 101 or a radiation information system for obtaining inspection request information. The external equipment 106 indicates, for example, an external system capable of DICOM communication, in general, typified by an RIS as a destination which obtains an imaging order, a PACS for saving and displaying obtained images, and a DICOM viewer. RIS is the abbreviation of Radiology Information System. PACS is the abbreviation of Picture Arching and Communication System. DICOM is the abbreviation of Digital Imaging and COmmunication in Medicine.

Upon receiving inspection request information from the RIS provided in the external equipment 106, the radiation imaging system images an object by using the radiation generation unit 103 and the sensor unit 102 and transmits inspection information including obtained images to the external equipment 106. For the sake of descriptive convenience, the following will describe an example of an arrangement in which the external equipment 106 is not included in the radiation imaging system and the radiation imaging system communicates with the external equipment 106 via a network. However, the external equipment 106 need not always be provided independently of the radiation imaging system, and may be implemented on the same apparatus on which the control unit 101 is implemented. In addition, this embodiment will exemplify the execution of radiation imaging as an example of a medical inspection. However, the embodiment may be applied to other types of medical inspections such as tomography and fundus examination.

(Functional Arrangement)

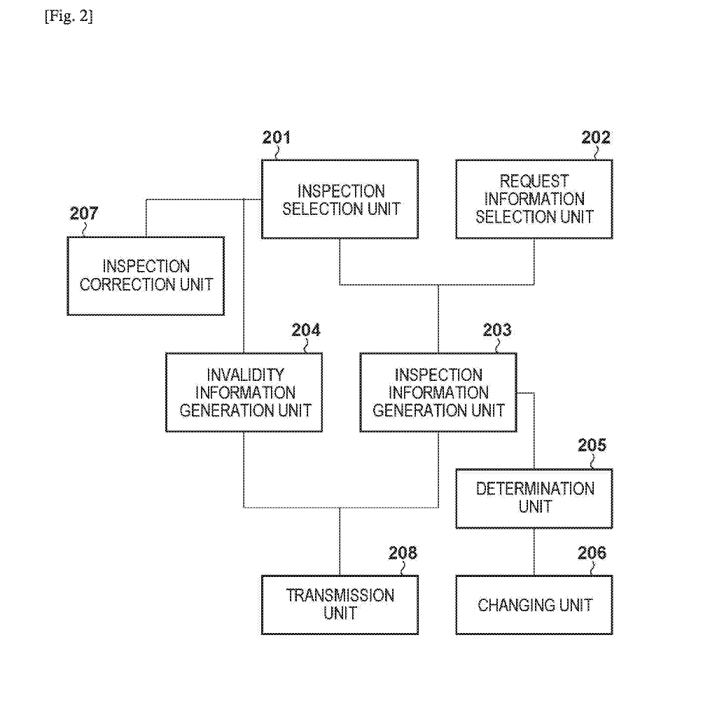

An example of the functional arrangement of the above control unit 101 will be described next with reference to FIG. 2. The following will exemplify a case in which each functional block is implemented as a function of the control unit 101 based on a computer program. However, at least some of these functional blocks may be implemented by other devices in the radiation imaging system.

FIG. 2 is a block diagram showing an example of the functional arrangement of the radiation imaging system according to the first embodiment of the present invention. As shown in FIG. 2, the control unit 101 includes as its functions, for example, an inspection selection unit 201, a request information selection unit 202, an inspection information generation unit 203, an invalidity information generation unit 204, a determination unit 205, a changing unit 206, an inspection correction unit 207, and a transmission unit 208.

The inspection selection unit (transmitted inspection selection unit) 201 is a functional block for designating inspection information about a wrongly executed inspection from the inspection information transmitted from the radiation imaging system to the external equipment 106 in accordance with an operation by the operator. The inspection selection unit 201 causes the display unit 104 to display, in a selectable form, the inspection information transmitted to the external equipment 106 and including the test image. Inspection errors include, for example, a mix-up between patients, an inspection content (for example, an imaging portion and laterality) error, and a mix-up between test images. The inspection selection unit 201 selects a transmitted inspection list displayed on a GUI on the display unit 104 with an input device such as the pointing device or keyboard of the operation unit 105. Inspection information is information for specifying each inspection. This embodiment will exemplify a case in which inspection information includes inspection dates, patient IDs (the identification information of subjects), patient names, the birth dates and sexes of the patients, imaging techniques (for example, imaging schemes, imaging portions, laterality, and imaging modes), and obtained images. However, inspection information is not limited to this. For example, the inspection information may not include part of the above information, such as obtained images or may include another information, for example, detailed profiles of patients (for example, previous diseases and addresses).

The request information selection unit (inspection request information selection unit) 202 is a functional block which selects inspection request information based on which an inspection should be performed. The request information selection unit 202 displays, in a selectable form on the display unit 104, a list of inspection request information obtained from the RIS provided in the external equipment 106, and prompts the operator to select displayed inspection request information with an input device such as the pointing device or keyboard of the operation unit 105. Inspection request information is information for requesting an inspection which is transmitted from the RIS in the external equipment 106 to the radiation imaging system. This embodiment will exemplify a case in which inspection request information includes receipt numbers, patient IDs, patient names, and specifying information for specifying imaging techniques. However, the embodiment is not limited to this example.

The inspection information generation unit 203 is a functional block which generates correct inspection information, based on which an inspection should have been executed, from the inspection information selected by the inspection selection unit 201 and the inspection request information selected by the request information selection unit 202.

The invalidity information generation unit 204 is a functional block which generates invalidity information with respect to an image or inspection included in the inspection information selected by the inspection selection unit 201. Invalidity information is information including a reason why the inspection is invalid and identification information for specifying an inspection or image to be made invalid. More specifically, invalidity information can be generated as, for example, Rejection Note defined in DICOM.

The determination unit (additional imaging necessity determination unit) 205 is a block which determines whether it is necessary to perform additional imaging with respect to the inspection information generated by the inspection information generation unit 203. This block determines the necessity of additional imaging based on the inspection information selected by the inspection selection unit 201 and the inspection request information selected by the request information selection unit 202. A more specific determination technique will be described later.

The changing unit (inspection state changing unit) 206 is a block which changes an inspection state to discriminate whether an inspection associated with the inspection information generated by the inspection information generation unit 203 requires additional imaging. The inspection correction unit (executed inspection correction unit) 207 is a functional block which sets, as an invalid inspection, the inspection selected by the inspection selection unit 201 as a wrong inspection, and saves the images included in the inspection as rejected images. The transmission unit (information transmission unit) 208 is a functional block which transmits, to the external equipment 106, the inspection information generated by the inspection information generation unit 203 and the invalidity information generated by the invalidity information generation unit 204.

(Processing Procedure)

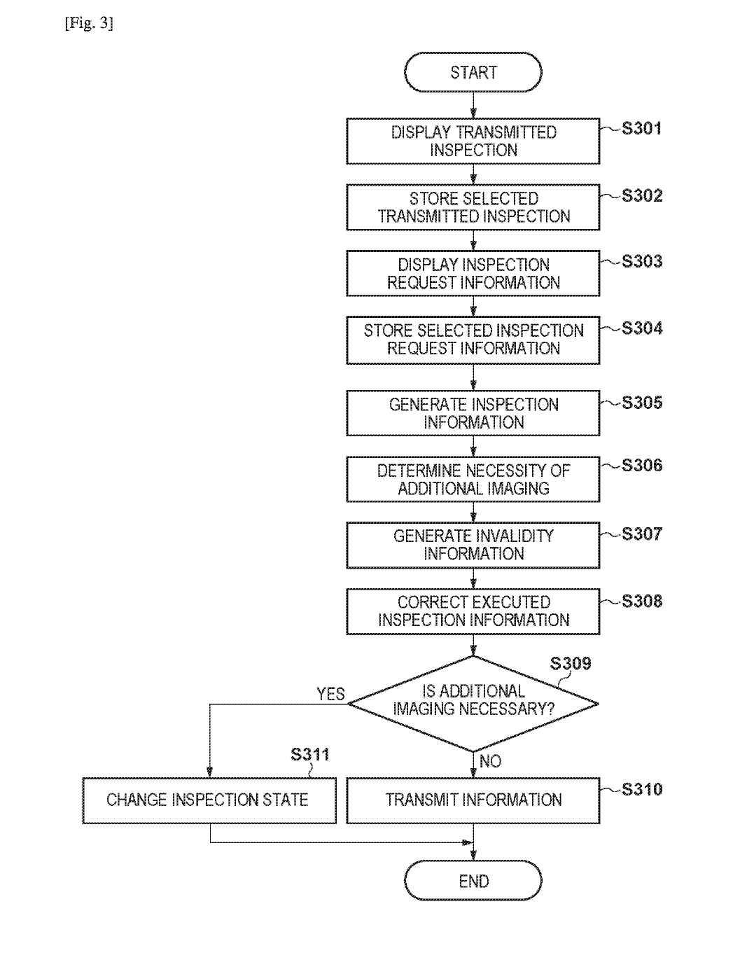

A processing procedure according to this embodiment will be described next with reference to FIG. 3. FIG. 3 is a flowchart showing a processing procedure executed by the radiation imaging system according to the embodiment. This flowchart shows a typical processing procedure from the step of causing the inspection selection unit 201 to select a correction target inspection from displayed transmitted inspections to the step of correcting inspection information. This procedure is based on the premise that there is an inspection which has been wrongly executed. Each step described below is executed under the control of the CPU (Central Processing Unit) 114.

In step S301, the inspection selection unit 201 displays an inspection externally transmitted from the radiation imaging system to the external equipment 106. The CPU then accepts the selection of inspection information including a mix-up between patients, an inspection content error, or the like. When, for example, the operator issues a request to display a transmitted inspection display screen, the control unit 101 reads the inspection information of inspections executed in the past from a DB (DataBase) or the like constructed in the nonvolatile storage device 115, and displays a list of inspection information in a selectable form on the display of the display unit 104.

When the operator selects the inspection information of an inspection wrongly executed from the displayed list of transmitted inspections, the inspection information selected in step S302 is stored in the RAM 110, the nonvolatile storage device 115, or the like.

In step S303, the request information selection unit 202 displays the inspection request information obtained from the RIS provided in the external equipment 106. The CPU then accepts the selection of inspection request information which should be associated with the content of the inspection information selected in step S301. For example, the radiation imaging system obtains inspection request information from the RIS in the external equipment 106 in accordance with clicking (selection) of a button or the like displayed on a GUI, and displays a list of obtained request information. When the operator selects desired request information from the displayed request information, the selected request information is stored in the RAM 110, the nonvolatile storage device 115, or the like in step S304.

In step S305, the inspection information generation unit 203 generates new inspection information based on the inspection request information stored in step S304. More specifically, the inspection information generation unit 203 generates inspection information including patient IDs and patient names set as the same values as those of the patient IDs and the patient names included in the inspection request information, with other parameters such as an inspection dates, imaging techniques, and obtained images being set to initial values (for example, NULL or the like). In addition, the inspection information generation unit 203 copies an image, of the images included in transmitted inspections, whose imaging technique matches that of the request information and associates the image as a new test image.

In step S306, the determination unit 205 determines whether the inspection generated in step S305 requires additional imaging. More specifically, if the imaging technique included in the transmitted inspection selected in step S301 is completely the same as that included in the inspection request information selected in step S303, the image included in the transmitted inspection may be set as a new test image. For this reason, the determination unit 205 determines that there is no need to perform additional imaging. In contrast to this, if the imaging technique included in the request information does not match the imaging technique for the transmitted test image, or transmitted images are insufficient with respect to the imaging techniques included in the request information even if the techniques match each other, the determination unit 205 determines that it is necessary to perform additional imaging.

In step S307, the invalidity information generation unit 204 generates invalidity information with respect to the inspection information stored in step S302. More specifically, the invalidity information generation unit 204 generates Rejection Note defined in DICOM with respect to the inspection stored in step S302. Rejection Note may include a reason for invalidity, for example, information indicating a correction request based on wrong selection of inspection request information.

In step S308, the CPU determines the inspection selected in step S302 as an invalid inspection, and determines all the obtained images included in the same inspection as rejected images. In step S309, the process branches based on the determination result obtained by the determination unit 205 in step S306. If the CPU determines that additional imaging is unnecessary (NO in step S309), the process shifts to step S310. The transmission unit 208 transmits the inspection information generated in step S305 and the invalidity information generated in step S307. If the CPU determines that additional imaging is necessary (YES in step S309), the process shifts to step S311. The inspection information generated in step S305 is marked as an additional imaging required inspection and saved in a database or the like. The processing is then terminated.

(GUI Examples)

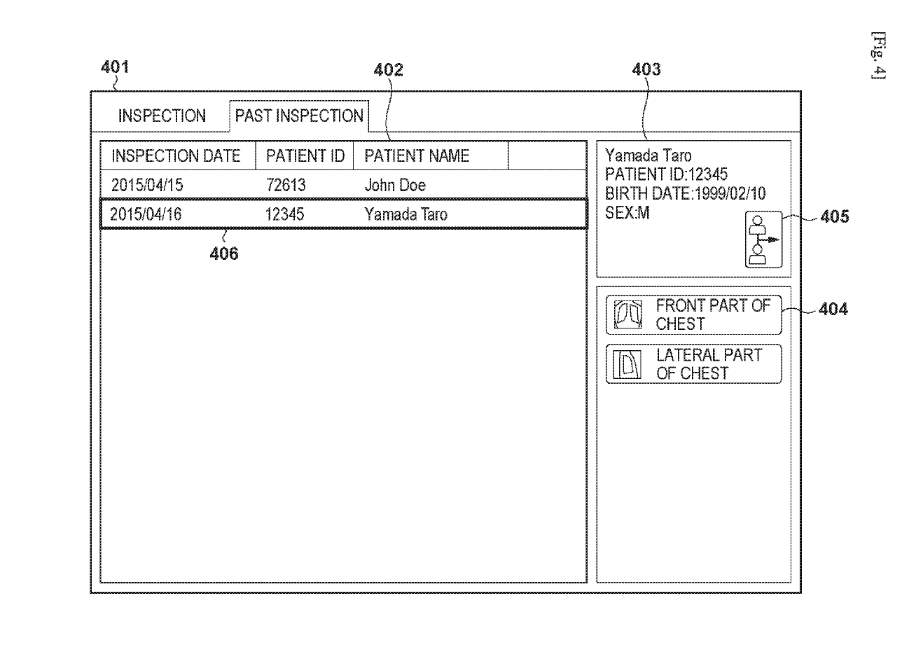

GUI examples in the radiation imaging system will be described next with reference to FIGS. 4, 5, 6, and 7. FIGS. 4 to 7 are views showing GUI examples displayed on the display unit 104 of the radiation imaging system.

FIG. 4 is a view showing an example of a transmitted inspection selection screen which displays a list of transmitted inspection information in a selectable form. The transmitted inspection selection screen is displayed in step S301 in FIG. 3. Referring to FIG. 4, the operator uses a transmitted inspection selection screen 401 and a transmitted inspection list display area 402 to select a transmitted inspection including an inspection error such as a mix-up between patients. Referring to FIG. 4, a frame 406 indicates a state in which “patient ID: 12345” and “patient name: Yamada Taro” are selected. A patient information display area 403 is an area for displaying the patient information of a selected transmitted inspection in more detail. An imaging technique display area 404 displays an image obtained in a selected inspection together with an imaging technique and a thumbnail image to allow the operator to check at a glance a specific image acquired by a specific technique. The example shown in FIG. 4 indicates that a “front part of chest” image and a “lateral part of chest” image were acquired from a patient (patient name: Yamada Taro) identified by “patient ID: 12345” at “inspection date: 2015/04/16”. A call button (inspection request information selection screen call button) 405 is used to display a screen for selecting inspection request information. When the call button 405 is selected by clicking on a pointing device or the like, a touch panel, or the like, the radiation imaging system calls up a screen for selecting inspection request information. The following is a case in which while the inspection information specified by the frame 406 is selected, the call button 405 is selected.

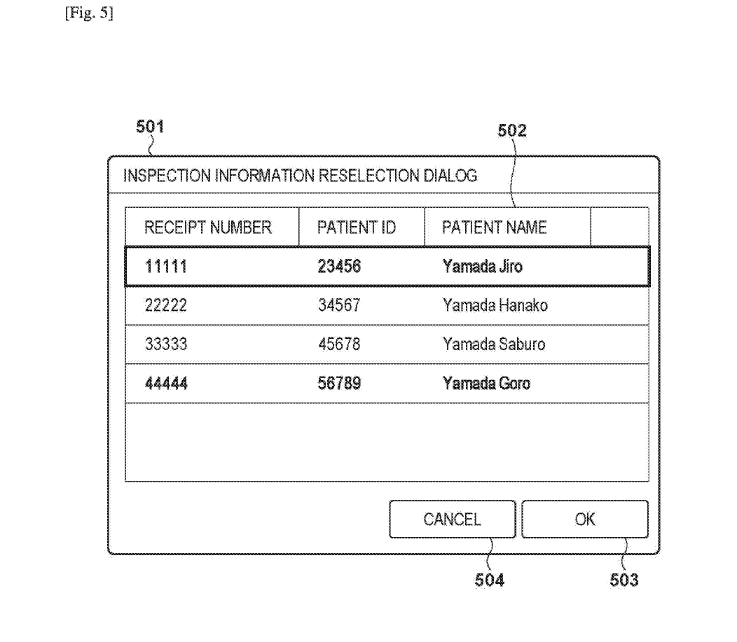

FIG. 5 is a view showing an example of an inspection request information selection screen called up by pressing the call button 405. The inspection request information selection screen is displayed in step S303 in FIG. 3. An inspection request information selection screen 501 is a screen for selecting inspection request information. This screen displays the inspection request information obtained from the RIS of the external equipment 106. Inspection request information to be displayed may be obtained at the time of displaying this screen or may be obtained in advance. An inspection request information list display area 502 is used to display a list of inspection request information obtained from the RIS of the external equipment 106 and prompt the operator to select inspection request information. FIG. 5 shows a state in which inspection request information including “receipt number: 11111”, “patient ID: 23456”, and “patient name: Yamada Jiro” is selected.

Note that in the inspection request information list display area 502, the inspection request information displayed in boldface indicates request information including the same imaging technique as that in the inspection selected on the transmitted inspection selection screen (FIG. 4). In the above case, referring to FIG. 4, an inspection corresponding to “patient ID: 12345” from which the “front part of chest” image and the “lateral part of chest” image are acquired is selected, and hence inspection request information including an imaging request for “front part of chest” or “lateral part of chest” is displayed in the inspection request information list display area 502. Referring to FIG. 5, request information of “receipt number: 11111” and “receipt number: 44444” corresponds to this information. Displaying request information including the same imaging technique as that in the inspection selected on the transmitted inspection selection screen so as to discriminate the request information from other request information in this manner makes it possible to easily identify inspection request information which is likely to be the inspection which should have been executed. In this embodiment, changing the thickness of characters to be displayed indicates that the corresponding imaging techniques are the same. However, any scheme can be used as long as it can identify information, of the request information displayed in the form of a list, which includes the same imaging technique as that in the inspection selected on the transmitted inspection selection screen. For example, it is possible to use a scheme of displaying graphics (graphical patterns) such as icons and marks or a scheme of displaying character information such as texts and labels.

An OK button 503 is used to collectively instruct the processing of confirming selected inspection request information, generating new inspection information, determining whether the generated inspection information requires additional imaging, generating invalidity information with respect to a transmitted inspection, and setting a transmitted test image as a rejected image. A cancel button 504 is used to cancel the selection of inspection request information and close this screen. The following is a case in which the OK button 503 is selected while the inspection request information of “patient ID: 23456” and “patient name: Yamada Jiro” is selected.

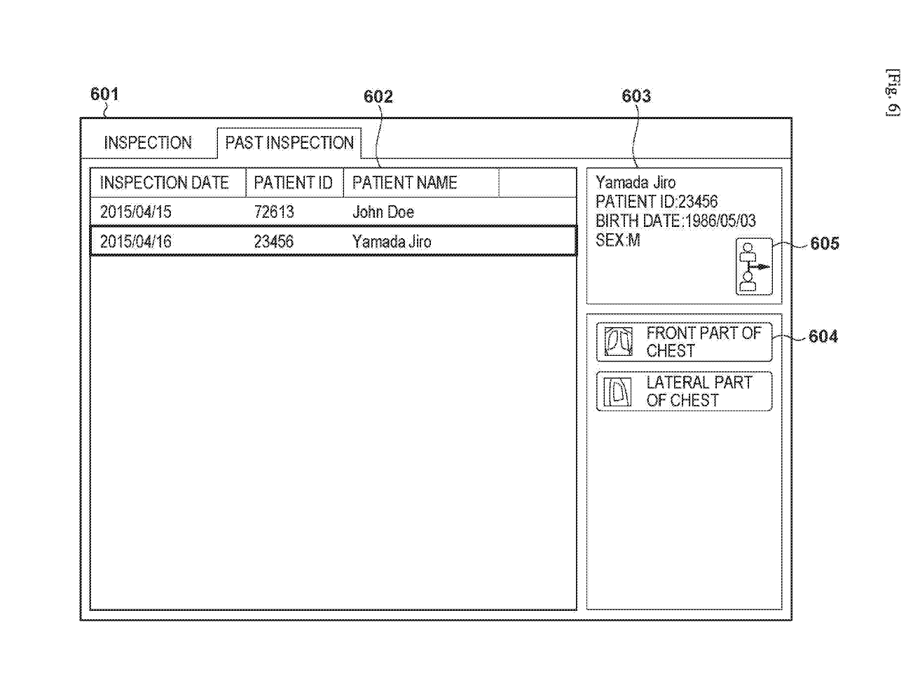

FIG. 6 is a transmitted inspection selection screen after the generation of new inspection information in FIG. 5, and shows a state in which inspection information is newly generated based on the inspection request information selected in FIG. 5. In the example shown in FIG. 6, the inspection information of “patient ID: 23456” and “patient name: Yamada Jiro” is newly generated, and the inspection information of “patient ID: 12345” and “patient name: Yamada Taro” in FIG. 4 is replaced by the inspection information of “patient ID: 23456”.

FIG. 5 shows the case in which the imaging techniques included in the transmitted inspection are completely the same as those included in the inspection request information, and hence no re-imaging is required (NO in step S309). For this reason, in this state, the processing has been executed up to the transmission of the test images in response to the selection of the OK button 503 in FIG. 5. That is, in response to clicking (selection) of the OK button 503, steps S304 to S310 in FIG. 3 have been automatically executed, and the transmitted inspection has been set as an invalid inspection. In this case, all the images included in this inspection can be set as rejected images. Note that areas 601 to 605 in FIG. 6 are identical to the areas 401 to 405 in FIG. 4.

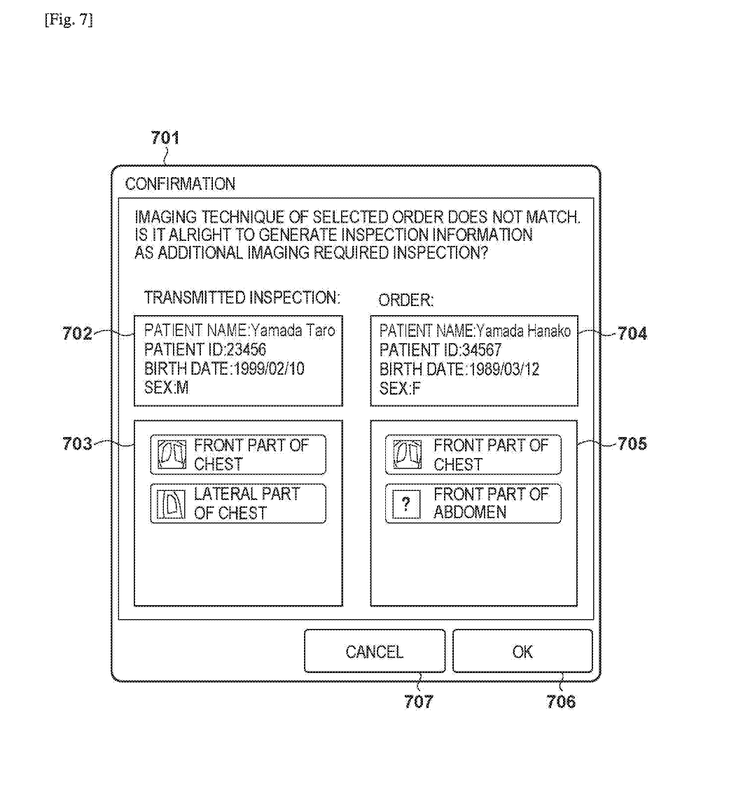

An example of a GUI in a case in which additional imaging is required (YES in step S309) will be described next with reference to FIG. 7. A confirmation screen 701 is displayed when the OK button 503 is selected upon selection of inspection request information including an imaging technique which does not match that included in the selected inspection information in FIG. 5. A transmitted inspection patient information display area 702 is an area for displaying the patient information of an inspection selected as a transmitted inspection. The example shown in FIG. 7 indicates that the inspection information of “patient ID: 12345” and “patient name: Yamada Taro” has already been transmitted.

A transmitted inspection imaging technique display area 703 is an area for displaying imaging techniques selected as those of a transmitted inspection. In the example shown in FIG. 7, “front part of chest” and “lateral part of chest” are displayed in the transmitted inspection imaging technique display area 703. This indicates that this inspection request information includes two obtained images, namely a “front part of chest” image and a “lateral part of chest” image.

An inspection request patient information display area 704 is an area for displaying the patient information of selected inspection request information. The example shown in FIG. 7 indicates that the inspection request information of “patient ID: 34567” and “patient name: Yamada Hanako” is received. An inspection request information imaging technique display area 705 is an area for displaying the imaging techniques included in the inspection request information. The example shown in FIG. 7 indicates that the inspection request information includes two imaging techniques, namely “front part of chest” and “front part of abdomen”. In this case, since the imaging technique “front part of chest” matches that included in the selected inspection information, the corresponding thumbnail image is displayed. However, since there is no obtained image corresponding to “front part of abdomen”, there is no associated image, and “?” is displayed to indicate that additional imaging is required. In this manner, this embodiment is configured to compare the imaging techniques for test images included in selected inspection information with the imaging techniques specified by the specifying information included in selected inspection request information to determine the necessity of an additional inspection. If it is determined that an additional inspection is required, display control is performed to cause the display unit 104 to display information indicating that an additional inspection is required. According to the embodiment, therefore, the user can grasp at a glance the necessity of an additional inspection.

An OK button 706 is used to issue an instruction to generate inspection information upon generating inspection information and setting a state in which additional imaging is required. A cancel button 707 is used to cancel the generation of inspection information and close the screen.

As described above, this radiation imaging system decides whether a test image which has been obtained in accordance with wrong inspection request information can be used as a test image which should have been obtained, and determines whether additional imaging is required for an inspection which should be done. This makes it possible to efficiently execute processing required to redo an inspection.

In addition, this embodiment generates new inspection information and transmits it to the external equipment 106 upon associating the test images included in selected inspection information with the identification information of a subject included in selected inspection request information. For this reason, even if a mix-up between patients or the like occurs, the user can easily correct inspection information.

Furthermore, if it is determined that additional inspection is required, new inspection information about inspection request information determined to require the additional inspection is transmitted after the execution of the additional inspection. That is, an inspection determined to require additional imaging is inhibited from being externally transmitted until necessary images are acquired. This can prevent the transmission of wrong inspection information.

Further transmitting invalidity information for invalidating selected inspection information to the external equipment 106 makes it possible to effectively invalidate inspection information including an inspection error. In this case, causing the display unit 104 to display inspection information including invalidity information so as to discriminate the inspection information from other inspection information makes it possible to easily discriminate other inspection information. Furthermore, it is possible to automatically classify an image including an inspection error as a rejected image by correcting a test image included in selected inspection information as a rejected image in accordance with the generation of new inspection information.

### Second Embodiment

The first embodiment has exemplified the case in which after correct inspection information is generated, any inspection information obtained by wrongly executing an inspection is not displayed. It is assumed that the operator may want to refer to a specific inspection which failed in the past. It is possible to make setting in advance to allow the operator to determine whether to leave an inspection which has been wrongly executed as a wrongly executed inspection or delete or hide it when correcting inspection information and to manage the wrongly executed inspection based on the setting. In this case, since a wrongly executed inspection is an inspection executed based on wrong patient information and the patient information is not correct with respect to an image, the image is not appropriate as a diagnosis image. It is therefore preferable to reduce the risk of diagnosis based on inappropriate images by performing control to inhibit any images included in inspections managed as wrongly executed inspections from being output from this radiation imaging system to the outside.

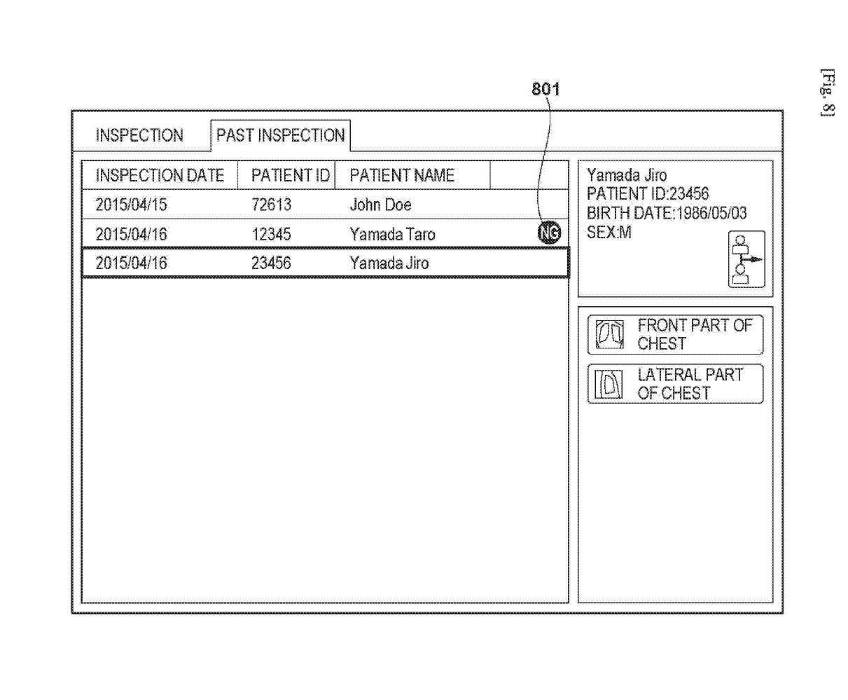

FIG. 8 shows an example of a transmitted inspection selection screen when setting is made to leave a wrongly executed inspection upon completion of the generation of inspection information. A wrongly executed inspection identification mark 801 is a mark indicating that this inspection was a wrongly executed inspection. That is, this mark indicates that invalidity information generated by an invalidity information generation unit 204 has already been transmitted with respect to this inspection. FIG. 8 shows that the inspection corresponding to “patient name: Yamada Taro” is a wrongly executed inspection. Any image included in an inspection indicated by this mark is handled as a rejected image, and setting can be made to allow the operator to refer to the image within this radiation imaging system but inhibit the image from being output to external equipment 106, as described in the first embodiment.

As described above, the risk of diagnosis based on inappropriate images can be reduced by making it possible to make setting to determine whether to hold an inspection determined to be invalid as a wrongly executed inspection or move the generated inspection information and delete or hide the original inspection information. Such risk can also be reduced by displaying a list of invalidity information while discriminating whether each invalidity information indicates a transmitted inspection or by displaying each inspection so as to allow discrimination of the inspection as an invalid inspection as a whole.

### Third Embodiment

When performing an inspection requiring additional imaging, the operator must perform re-imaging upon summoning the patient, thus imposing a heavy burden on the patient. For this reason, the operator needs to treat the patient more politely than usual. It is therefore preferable to allow the operator to identify normal inspections and inspections determined as inspections requiring additional imaging because of wrongly executed inspections so as to grasp in advance before the start of an inspection whether the inspection requires additional imaging.

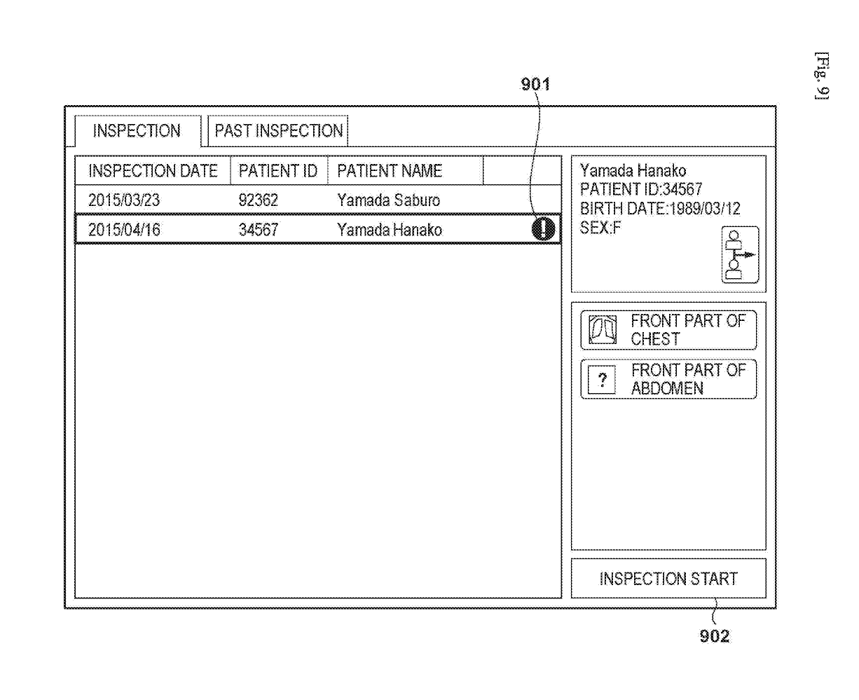

FIG. 9 shows an example of an incomplete inspection display screen upon selection of an instruction button 706 in FIG. 7. An additional imaging required inspection identification mark 901 is a mark for identifying the corresponding inspection determined as an inspection requiring additional imaging because of a wrongly executed inspection. FIG. 9 shows that an inspection corresponding to “patient name: Yamada Hanako” is an additional imaging required inspection. An inspection start button 902 is a button to be clicked (selected) to resume an inspection selected as an incomplete inspection.

In this embodiment, additional imaging required inspection identification mark is displayed to allow the operator to identify an inspection requiring additional imaging. However, this information may be displayed in the form of character information using texts or labels. That is, it is possible to use any scheme which can identify an inspection as an additional imaging required inspection from normal inspections and wrongly executed inspections of the inspections displayed in the form of a list.

As described above, in each embodiment according to the present invention, correct inspection information is generated by selecting a wrongly executed inspection and inspection request information of an inspection which should have been executed. At this time, if imaging techniques for test images obtained by wrong execution sufficiently match the imaging techniques included in inspection request information, it is determined that the inspection does not require additional imaging. If the techniques do not match each other, the corresponding inspection is determined as an inspection requiring additional imaging, and hence is managed as an additional imaging required inspection. This makes it possible to easily correct inspection information from the radiation imaging system and reduce the operation load on the operator even if the operator notices an error in an inspection transmitted to external equipment.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-041488, filed Mar. 3, 2016, which is hereby incorporated by reference herein in its entirety.

1. An information processing apparatus which controls a medical inspection based on inspection request information received from external equipment, the apparatus comprising:
a first display control unit configured to cause a display unit to display inspection information including a test image in a selectable form, which is transmitted to the external equipment;
a second display control unit configured to cause the display unit to display inspection request information including specifying information for specifying an imaging technique in a selectable form, which is received from the external equipment;
a determination unit configured to determine necessity of an additional inspection by comparing an imaging technique for a test image included in inspection information selected on display performed by the first display control unit with an imaging technique specified by the specifying information included in inspection request information selected on display performed by the second display control unit; and
a third display control unit configured to cause the display unit to display information indicating that an additional inspection is required, when the determination unit determines that an additional inspection is required.

2. The apparatus according to claim 1, further comprising:
a generation unit configured to generate new inspection information by associating a test image included in the selected inspection information with identification information of a subject included in the selected inspection request information; and
a transmission unit configured to transmit the new inspection information to the external equipment.

3. The apparatus according to claim 2, wherein if the determination unit determines that an additional inspection is required, the transmission unit transmits the new inspection information about inspection request information determined to require the additional inspection after the additional inspection is performed.

4. The apparatus according to claim 2, further comprising a correction unit configured to correct as a rejected image a test image included in the selected inspection information in accordance with generation of new inspection information by the generation unit.

5. The apparatus according to claim 1, further comprising a transmission unit configured to transmit, to the external equipment, invalidity information for invalidating inspection information which corresponds to the selected inspection information and is managed by the external equipment.

6. The apparatus according to claim 5, wherein the first display control unit causes the display unit to display inspection information to which the invalidity information is transmitted so as to discriminate the inspection information from other inspection information.

7. The apparatus according to claim 1, wherein the second display control unit causes the display unit to display inspection request information of an information about an imaging technique which matches an imaging technique for an inspection image included in inspection information selected on display by the first display control unit so as to discriminate the inspection request information from other inspection request information.

8. An information processing apparatus which controls a medical inspection based on inspection request information received from external equipment, the apparatus comprising:
a first display control unit configured to cause a display unit to display inspection information including a test image in a selectable form, which is transmitted to the external equipment;
a second display control unit configured to cause the display unit to display inspection request information including specifying information for specifying an imaging technique in a selectable form, which is received from the external equipment;
a determination unit configured to determine necessity of an additional inspection by comparing an imaging technique for a test image included in inspection information selected on display performed by the first display control unit with an imaging technique specified by the specifying information included in inspection request information selected on display performed by the second display control unit; and
a transmission unit configured to transmit, to the external equipment, invalidity information for invalidating inspection information which corresponds to the selected inspection information and is managed by the external equipment, if the determination unit determines that an additional inspection is required.

9. An information processing method for an information processing apparatus which controls a medical inspection based on inspection request information received from external equipment, the method comprising:
causing a first display control unit configured to cause a display unit to display inspection information including a test image in a selectable form, which is transmitted to the external equipment;
causing a second display control unit configured to cause the display unit to display inspection request information including specifying information for specifying an imaging technique in a selectable form, which is received from the external equipment;
causing a determination unit configured to determine necessity of an additional inspection by comparing an imaging technique for a test image included in inspection information selected on display performed in the causing the first display control unit with an imaging technique specified by the specifying information included in inspection request information selected on display performed in the causing the second display control unit; and
causing a third display control unit configured to cause the display unit to display information indicating that an additional inspection is required, when it is determined in the causing the determination unit that an additional inspection is required.

10. A non-transitory computer-readable storage medium storing a computer program for causing a computer to function as each unit of an information processing apparatus which controls a medical inspection based on inspection request information received from external equipment, the information processing apparatus comprising:
a first display control unit configured to cause a display unit to display inspection information including a test image in a selectable form, which is transmitted to the external equipment;
a second display control unit configured to cause the display unit to display inspection request information including specifying information for specifying an imaging technique in a selectable form, which is received from the external equipment;
a determination unit configured to determine necessity of an additional inspection by comparing an imaging technique for a test image included in inspection information selected on display performed by the first display control unit with an imaging technique specified by the specifying information included in inspection request information selected on display performed by the second display control means; and
a third display control unit configured to cause the display unit to display information indicating that an additional inspection is required, when the determination unit determines that an additional inspection is required.
